(19) United States
(12) Patent Application Publication
(10) Pub. No.: US 2021/0353301 A1
(43) Pub. Date: Nov. 18, 2021

(54) METHODS FOR PREVENTING RETROPULSION OF CONCRETIONS AND FRAGMENTS DURING LITHOTRIPSY

(71) Applicant: Genzyme Corporation, Cambridge, MA (US)

(72) Inventors: Jean-Marie Vogel, Cambridge, MA (US); James A. Wilkie, Molrose, MA (US)

(21) Appl. No.: 17/187,409

(22) Filed: Feb. 26, 2021

Related U.S. Application Data
(63) Continuation of application No. 15/282,186, filed on Sep. 30, 2016, now abandoned.
Application No. 15/282,186 is a continuation of application No. 11/864,118, filed on Sep. 28, 2007, now abandoned.
(60) Provisional application No. 60/848,244, filed on Sep. 29, 2006.

(51) Int. Cl. A61B 17/12; A61B 17/22; A61B 18/26; A61M 5/00

(52) U.S. Cl. CPC A61B 17/12186; A61B 17/12099; A61B 17/12195; A61B 17/22012; A61B 2017/22067; A61B 17/12109; A61B 17/12022; A61M 5/007; A61B 18/26

(57) ABSTRACT

One aspect of the present invention provides a method for the treatment of lithiasis, which mitigates the risk of damage to surrounding body tissue when removing a calculi (e.g., biological concretions, such as urinary, biliary, and pancreatic stones) that obstructs or may otherwise be present within a body's anatomical lumen. In one embodiment, the instant invention provides a method of using a polymer plug to occlude a lumen distal to a calculi, whereby calculi fragments resulting from lithotripsy are prevented from traveling up the lumen. In certain embodiments, a dual lumen catheter is utilized to inject two solutions proximal to the calculi, the mixing of said solutions causing a polymer plug to form.

### RELATED APPLICATIONS

This application is a continuation of U.S. application Ser. No. 15/282,186, filed on Sep. 30, 2016, which is a continuation of U.S. application Ser. No. 11/864,118, filed on Sep. 28, 2007, which claims the benefit of priority to U.S. Provisional Application No. 60/848,244, filed on Sep. 29, 2006. The entire teachings of the above application(s) are incorporated herein by reference.

### BACKGROUND OF THE INVENTION

Lithiasis is a common human ailment characterized by concretions or “stones” formed within a passage of the human body. While stones have been documented in just about every passage within the body, kidney stones (nephrolithiasis) and gallstones (cholelithiasis) are the most common. Regardless of its location, however, a stone is typically an extremely hard and unyielding mass which blocks the passage (e.g., lumen) in which it presents.

Concretions in the urinary tract or kidneys usually arise because of the breakdown of a delicate balance in the body. Specifically, the kidneys must conserve water to function, but they must excrete materials that have a low solubility. These opposing requirements must be balanced during adaptation to diet, climate and activity. The problem is mitigated to some degree because urine contains substances that inhibit crystallization of stone-forming minerals. However, when urine becomes supersaturated with insoluble materials, because excretion rates are excessive and/or because water conservation is extreme, crystals form and may grow and aggregate to form a stone.

Although small crystals are readily voided from the kidney with urine, the larger stones frequently become dislodged from the kidney and enter the ureter or occlude the uretero-pelvic junction, causing obstruction and pain. Although some stones can ultimately traverse the ureter, their passage typically produces pain and bleeding. Usually, the pain is so severe that narcotic drugs are needed for its control.

Removal of stones from the kidneys or urinary tract can be effected medically, mechanically or surgically. A well-known surgical approach involves passing a flexible basket in a retrograde manner up the ureter from the bladder, and using the basket to capture the stones. However, the baskets require post-capture removal and only work well for medium-sized stones. Surgery has also been used to remove kidney stones, especially so-called staghorn stones which get lodged in the ureter.

Another surgical technique, percutaneous ultrasonic lithotripsy, requires the passage of a rigid cystoscopy-like instrument in the renal pelvis through a small incision in the flank whereupon stones are broken up by a small ultrasound transducer and then removed directly. Another surgical technique is laser lithotripsy via a ureteroscope. All of these procedures, which can be quite painful, are elaborate and expensive, and they do not always result in complete removal of the stones and fragments. One non-invasive technique, known as extracorporeal lithotripsy, entails transmission of high-intensity shock waves from outside the body to fragment the stones within the body. The resulting stone fragments are then voided with urine.

Stents have also been used to decompress ureteral obstructions, ensuring that urine drains from the kidney to the bladder. It was recognized that placement of a stent within the ureter could help small stones and stone fragments to transit the ureter. In a typical procedure involving a stent, a guide wire is passed through the ureter to the renal pelvis. A hollow, flexible, cylindrical stent is then advanced with a pusher over the guide wire. The guide wire and pusher are then extracted from the stent and the body, leaving an open lumen for urine to pass through. However, because the lumen defined by the cylindrical stent is even smaller than the ureter itself, all but the smallest stones and sludge are precluded from passing through. However, in many cases, stone fragments often block the open stent passageway.

All urologists who perform ureteroscopy for stone disease have had the experience of watching helplessly as a distal or proximal ureteral stone migrates cephalad, just out of reach or sight. Retrograde stone migration results in a longer operating time, more-invasive endoscopy, and an increase in residual stones and the need for secondary procedures, leading to higher morbidity and greater expense. With ureteroscopy now recommended as the preferred treatment modality for upper and lower ureteral stones, the problem of intraprocedural stone migration is magnified.

The distal ureteral stone (i.e., at or below the iliac vessels) usually causes some proximal ureteral dilatation. Dislodgement of the stone by the ureteroscope or by irrigation, a laser burst, pulsation of a pneumatic lithotriptor, or the spark of an electrohydraulic electrode can propel the stone cephalad, requiring a change from semirigid to flexible ureteroscopy, stenting, or a secondary procedure. A seemingly straightforward distal ureteral stone can rapidly deteriorate into a complicated problem. Data published by endourology specialists indicate that proximal migration requiring a secondary procedure occurs in 4-5% of distal ureteral stone cases; however, the percentage of stones that migrate in general practice is probably significantly higher. Furthermore, published data do not reflect migrating calculi that are successfully treated at the same sitting but require more-invasive procedures, such as an otherwise unnecessary stent or the use of a flexible ureteroscope (approximately US$500/use). Calculi in the upper ureter (i.e., above the iliac vessels) are even more likely to migrate cephalad during ureteroscopy. Even the Mayo Clinic group reported successful treatment of only 72% of proximal ureteral stones. Results in the average urologist's hands are probably not as good. A group from Berlin reported migration in greater than 40% of proximal ureteral stones using a pneumatic lithotriptor, and concluded that the pneumatic device should not be used for mid or proximal ureteral stones. With over 7,000 pneumatic lithotriptors in use, this represents a significant problem. A remarkable solution to this problem is described herein.

### SUMMARY OF THE INVENTION

One aspect of the present invention provides an approach to the treatment of lithiasis. Importantly, the present invention mitigates the risk of damage to surrounding body tissue when removing a calculi (e.g., biological concretions, such as urinary, biliary, and pancreatic stones) which obstructs or may otherwise be present within a body's anatomical lumen. Remarkably, the present invention improves significantly the treatment of lithiasis, while simultaneously reducing the risk of tissue damage and decreasing the procedure time. Importantly, the present invention prevents retropulsion of fragments during lithotripsy.

In one embodiment, the instant invention provides a method of using a polymer plug to occlude a lumen distal to a calculi, whereby calculi fragments resulting from lithotripsy are prevented from traveling up the lumen. In one embodiment the method is used as an alternative to conventional lithotripsy. In certain embodiments, a dual lumen catheter is utilized to inject two solutions proximal to the calculi, the mixing of said solutions causing a polymer plug to form.

Importantly, the inventive compositions and methods have distinct advantages over the materials currently on the market (such as Boston Scientific's Stone Cone and COOK's N-Trap). While all products prevent, to some degree, forward stone migration, the invention's unique design makes it ideal for releasing stones which are too large for extraction, and for preventing scattering of stone fragments (including stones less than 1 mm in diameter). In addition, unlike other approaches, in the inventive approach there is nothing placed in front of the stone; therefore, there is no interference with the fragmenting procedure. Finally, in certain embodiments, the robustness of the compositions used, which cannot be cut by a laser, provides an additional advantage.

### BRIEF DESCRIPTION OF FIGURES

FIGS. 1 and 2 depict various steps in a method of preventing retrograde migration of a concretion (e.g., stone) during intracorporeal lithotripsy. Key: [i] position catheter for injection behind concretion; [ii] inject composition of the invention to form a plug; [iii] retract catheter to free operating field; [iv] proceed with lithotripsy; [v] the plug prevents the migration of the fragments formed during lithotripsy; and [vi] irrigation with saline to dissolve the plug.

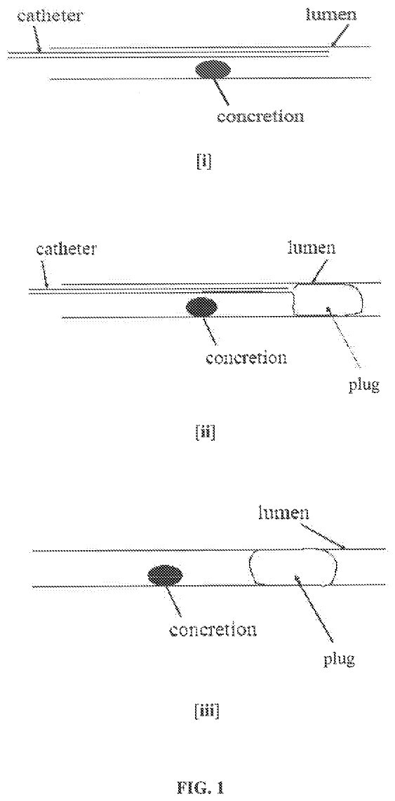

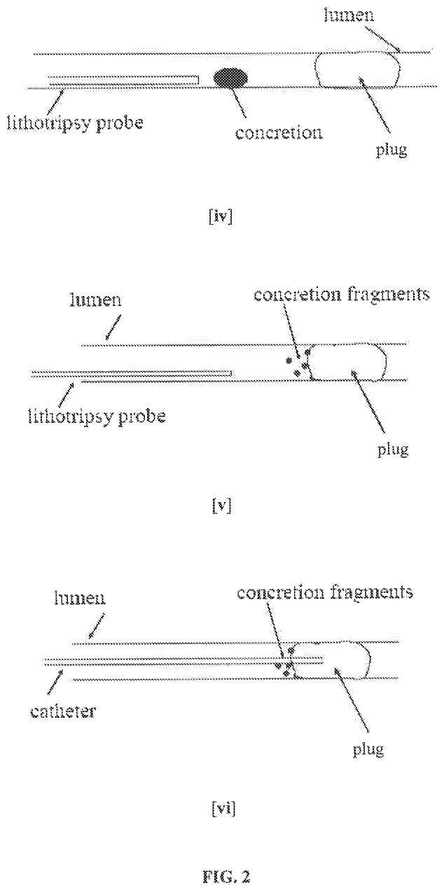

### DETAILED DESCRIPTION OF THE INVENTION

### Overview

The present invention improves significantly the success rate of lithotripsy and reduces the risk of tissue damage, by injecting a temporary plug behind a concretion (intracorporeal lithotripsy). The present invention mitigates the risk of damage to surrounding body tissue when performing lithotripsy to remove material (e.g., biological concretions, such as urinary, biliary, and pancreatic stones) which may obstruct or otherwise be present within the body's anatomical lumens.

One aspect of the present invention relates to injecting at least one composition (in certain embodiments two compositions) into a lumen, thereby forming a plug and preventing the migration of a concretion, or its fragments, during extracorporeal or intracorporeal lithotripsy. In one embodiment, the invention prevents the upward migration of concretion fragments generated during a fragmentation procedure. In certain embodiments, the lumen is cleared by rinsing with saline, which dissolves the plug. Dissolution and flushing of the dissolved plug also flushes the concretion fragments out of the lumen. In certain embodiments, the compositions used have no tissue-adhesive properties; i.e., they do not irreversibly bond to the lumen in which they are deployed. Also, because the material undergoes a phase change only under specific conditions, the material does not “cure” in situ.

Importantly, the invention also enables repeated or continuous application of energy to a concretion, and its resulting fragments, or other biological and non-biological/foreign material, while minimizing trauma to the surrounding tissue. The present invention improves significantly the success rate of lithotripsy, reduces the risk of tissue damage, and decreases time required for the procedure.

### Definitions

For convenience, certain terms employed in the specification, exemplification, and appended claims are collected here.

The articles “a” and “an” are used herein to refer to one or to more than one (i.e., to at least one) of the grammatical object of the article. By way of example, “an element” means one element or more than one element.

The term “contrast-enhancing” refers to materials capable of being monitored during injection into a mammalian subject by methods for monitoring and detecting such materials, for example by radiography or fluoroscopy. An example of a contrast-enhancing agent is a radiopaque material. Contrast-enhancing agents including radiopaque materials may be either water soluble or water insoluble. Examples of water soluble radiopaque materials include metrizamide, iopamidol, iothalamate sodium, iodomide sodium, and meglumine. Examples of water insoluble radiopaque materials include metals and metal oxides such as gold, titanium, silver, stainless steel, oxides thereof, aluminum oxide, zirconium oxide, etc.

As used herein, the term “polymer” means a molecule, formed by the chemical union of two or more oligomer units. The chemical units are normally linked together by covalent linkages. The two or more combining units in a polymer can be the same, in which case the polymer is referred to as a homopolymer. They can be also be different and, thus, the polymer will be a combination of the different units; these polymers are referred to as copolymers.

As used herein, “crosslinking” is when individual polymer chains are linked together by covalent bonds (“chemical crosslinking”) or ionic bonds (“ionic crosslinking”) to form a three dimensional network. In certain polymers this kind of process has the effect of producing a gel.

The term “biocompatible”, as used herein, refers to having the property of being biologically compatible by not producing a toxic, injurious, or immunological response in living tissue. The term “non-tissue adhesive”, as used herein denotes a substance (e.g., a polymer plug) does not adhere to biological tissue.

“Gelatin” as used herein refers to a protein product produced by partial hydrolysis of collagen extracted from skin, bones, cartilage, ligaments, etc. In gelatin, the natural molecular bonds between individual collagen strands are broken down into a form that rearranges more easily. Gelatin melts when heated and solidifies when cooled again. Together with water it forms a semi-solid colloidal gel.

“Alginic acid” as used here in is a naturally occurring hydrophilic colloidal polysaccharide obtained from the various species of brown seaweed (Phaeophyceae). It occurs in white to yellowish brown filamentous, grainy, granular or powdered forms. It is a linear copolymer consisting mainly of residues of β-1,4-linked D-mannuronic acid and α-1,4-linked L-glucuronic acid. These monomers are often arranged in homopolymeric blocks separated by regions approximating an alternating sequence of the two acid monomers, as shown below:

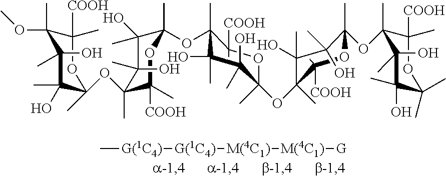

The formula weight of the structural unit is 176.13 (theoretical; 200 is the actual average). The formula weight of the macromolecule ranges from about 10,000 to about 600,000 (typical average).

“Sodium alginate” and “potassium alginate” are salts of alginic acid. For example, “potassium alginate” is shown below:

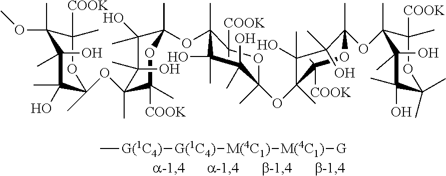

“Gellan gum” is a high molecular weight polysaccharide gum produced by a pure culture fermentation of a carbohydrate by Pseudomonas elodea, purified by recovery with isopropyl alcohol, dried, and milled. The high molecular weight polysaccharide is principally composed of a tetrasaccharide repeating unit of one rhamnose, one glucuronic acid, and two glucose units, and is substituted with acyl (glyceryl and acetyl) groups as the O-glycosidically-linked esters. The glucuronic acid is neutralized to a mixed potassium, sodium, calcium, and magnesium salt. It usually contains a small amount of nitrogen containing compounds resulting from the fermentation procedures. It has a formula weight of about 500,000. “Sodium gellan” and “potassium gellan” are salts of gellan gum. A gel sol transition occurs at about 50° C. depending on concentration.

Carboxymethylcellulose (CMC) is a polymer derived from natural cellulose. Unlike cellulose, CMC is highly water-soluble. The CMC structure is based on the β-(1,4)-D-glucopyranose polymer of cellulose. Different preparations may have different degrees of substitution, but it is generally in the range 0.6-0.95 derivatives per monomer unit, as shown below:

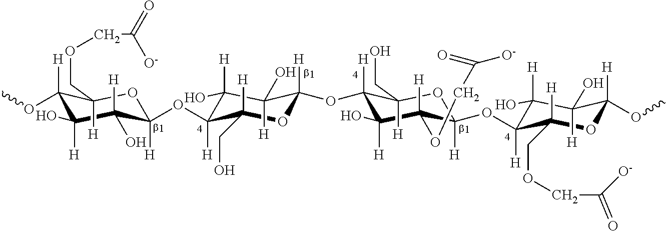

CMC molecules are somewhat shorter, on average, than native cellulose with uneven derivatization giving areas of high and low substitution. This substitution is mostly 2-O- and 6-O-linked, followed in order of importance by 2,6-di-O- then 3-O-, 3,6-di-O-, 2,3-di-O-lastly 2,3,6-tri-O-linked. It appears that the substitution process is a slightly cooperative (within residues) rather than random process giving slightly higher than expected unsubstituted and trisubstituted areas. CMC molecules are most extended (rod-like) at low concentrations but at higher concentrations the molecules overlap and coil up and then, at high concentrations, entangle to become a gel. Increasing ionic strength and reducing pH both decrease the viscosity as they cause the polymer to become more coiled. The average chain length and degree of substitution are of great importance; the more-hydrophobic lower substituted CMCs are thixotropic but more-extended higher substituted CMCs are pseudoplastic. At low pH, CMC may form cross-links through lactonization between carboxylic acid and free hydroxyl groups.

“Poly vinyl alcohol” (PVA) is a water soluble polymer synthesized by hydrolysis of a poly vinyl ester such as the acetate and used for preparation of fibers. PVA a thermoplastic that is produced from full or partial hydrolysis of vinyl ester such as vinyl acetate resulting in the replacement of some or all of the acetyl groups with hydroxyl groups. For example:

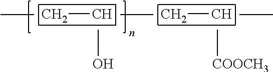

In certain embodiments polyvinyl alcohol (PVA) is a synthetic resin produced by polymerisation of vinyl acetate (VAM) followed by hydrolysis of the polyvinyl acetate (PVAc) polymer. The degree of polymerisation determines the molecular weight and viscosity in solution. The degree of hydrolysis (saponification) signifies the extent of conversion of the Polyvinyl Acetate to the Polyvinyl Alcohol For example n (Degree of Hydrolysis) may be in the range of about 68.2 to about 99.8 mol. % and the MW (Weight Average Molecular Weight) may range from about 10.000 to about 190,000.

Hyaluronic acid (HA) is a polymer composed of repeating dimeric units of glucuronic acid and N-acetyl glucosamine. It may be of extremely high molecular weight (up to several million daltons) and forms the core of complex proteoglycan aggregates found in extracellular matrix. HA is comprised of linear, unbranching, polyanionic disaccharide units consisting of glucuronic acid (GlcUA) an N-acetyl glucosamine (GlcNAc) joined alternately by β-1-3 and β-1-4 glycosidic bonds (see below). It is a member of the glycosaminoglycan family which includes chondroitin sulphate, dermatin sulphate and heparan sulphate. Unlike other members of this family, it is not found covalently bound to proteins.

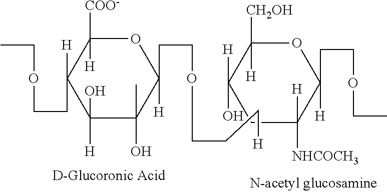

When incorporated into a neutral aqueous solution hydrogen bond formation occurs between water molecules and adjacent carboxyl and N-acetyl groups. This imparts a conformational stiffness to the polymer, which limits its flexibility. The hydrogen bond formation results in the unique water-binding and retention capacity of the polymer. It also follows that the water-binding capacity is directly related to the molecular weight of the molecule. Up to six liters of water may be bound per gram of HA.

HA solutions are characteristically viscoelastic and pseudoplastic. This rheology is found even in very dilute solutions of the polymer where very viscous gels are formed. The viscoelastic property of HA solutions which is important in its use as a biomaterial is controlled by the concentration and molecular weight of the HA chains. The molecular weight of HA from different sources is polydisperse and highly variable ranging from 10⁴ to 10⁷ Da. The extrusion of HA through the cell membrane as it is produced permits unconstrained polymer elongation and hence a very high molecular weight molecule.

The term “concretion” denote one or more masses or nodules of solid matter formed by growing together, by congelation, condensation, coagulation, induration, etc. Common synonyms, for example, are calculi, stones, clots, tones or lumps. Often, in an organism a concretion is a hard lump of mineral salts found in a hollow organ or duct. In one embodiment, concretion refers to stone-like objects found within an organ (e.g., the kidneys) of an organism.

The term “lumen” denotes the space enclosed by a tube-like structure or hollow organ, such as inside an artery, a vein, a kidney, a gall bladder, a ureter, a urinary bladder, a pancreas, a salivary gland, a small intestine or a large intestine (i.e., an opening, space, or cavity in a biological system). A lumen has an “inlet” and an “outlet” based on the direction of the flow of materials through the lumen. As used here “upstream” from a given object in a lumen means between said object and the inlet of the lumen; “downstream” from a given object in a lumen means between said object and the outlet of the lumen.

“Lithotripsy” as used herein refers to any procedure, surgery or technique that fragments or breaks up a stone. Lithotripsy also includes procedures such as percutaneous nephrolithotmy.

“Lithiasis” as used herein refers to a common human ailment characterized by concretion or “stones” formed within a passage or lumen of a human.

### Concretions

Concretions can develop in certain parts of the body, such as in the kidneys, pancreas, ureter and gallbladder. It is not uncommon for biological concretions to be referred to as calculi or stones, especially when they are composed of mineral salts. For example, concretions formed in the biliary system are called gallstones. Those that form in the bladder are also known as vesical calculi or bladder stones, and cystoliths. Concretions occurring in the kidney are often called kidney stones. Concretions can also occur in the ureter, where they are usually the result of the passage of one originating in the kidney. Concretions of the urinary bladder; also known as vesical calculi or bladder stones, and cystoliths. It is also possible to observe the presence of calculi in a salivary ducts or glands.

There are four main types of concretions observed biologically. The majority of concretions, about 75%, are calcium-containing, composed of calcium oxalate, sometimes mixed with calcium phosphate. Another 15% are composed of magnesium ammonium phosphate; these calculi are often referred to as “triple stones” or struvite stones. The bulk of the remaining stones are made up of uric acid or cystine. When these calculi are too large to pass spontaneously, medical intervention is often needed.

### Lithotripsy

Larger biological concretions often need to be shattered because their size prohibits non-surgical removal from the body. This procedure is known as lithotripsy. Shattering a concretion (by, for example, light, chemical, or physical energy) will disperse the resulting fragments from the original location of the concretion. It is important to remove all the fragments, as fragments that are not removed from the body can form the nuclei for the formation of new concretions. This process is made difficult by the fact that often the shattering process can cause fragments to move into inaccessible or unknown areas of the body thus preventing or interfering with the capture and removal of the fragments.

Intracorporeal lithotripsy utilizes a probe advanced with the aim of endoscope and positioned in proximity to the concretion. The energy, required for fragmentation is transferred through the probe to the concretion and the treatment process is visualized during fragmentation. The mode of energy transfer may be different and accordingly the intracorporeal lithotripsy techniques are divided into following groups: ultrasonic, laser, electro-hydraulic and mechanic/ballistic impact.

The last group comprises, for example, detonating an explosive near the concretion and causing the shock wave generated by the explosion to act directly upon the concretion and crush it into pieces. An example of such technique is disclosed in U.S. Pat. No. 4,605,003, referring to a lithotriptor comprising an inner tube inserted within an outer slender tube and provided with an explosive layer or a gas-generating layer. By the blasting of the explosive layer or the gas-generating layer, the outer slender tube or the inner tube is caused to collide with the stone and crush it.

An example of mechanical impact technique can be found in U.S. Pat. No. 5,448,363 in which is disclosed an endoscopic lithotriptor provided with a hammer element to periodically strike the concretion. The hammer element is pneumatically driven by a linear jet of air causing it to swing through an arc about a pivot to impact an anvil. There are known also many other patents, disclosing lithotriptors, which operation is based on mechanic/ballistic principle, e.g., U.S. Pat. No. 5,722,980 and U.S. Pat. No. 6,261,298.

An example of laser technique is described in U.S. Pat. No. 4,308,905, concerning multi-purpose lithotriptor, equipped with laser light-conducting fibers, through which the energy required for crushing the concretion is conducted.

Ultrasonic technique is relatively popular and because of its safety and usefulness is widely accepted. According to this principle ultrasound probe emits high-frequency ultrasonic energy that has a disruption effect upon direct exposure to the concretion. Direct contact of the probe tip and stone is essential for effectiveness of ultrasonic lithotripsy. This technique is implemented in many lithotriptors, e.g., as described in U.S. Pat. No. 6,149,656.

In addition there is electro-hydraulic technique, which utilizes electric discharge, ignited between two electrodes disposed within the probe and producing shock wave, expanding towards the concretion through liquid phase, which surrounds the concretion. In the literature electro-hydraulic lithotripsy is defined as the oldest form of “power” lithotripsy. The electro-hydraulic lithotriptor releases high-energy impulse discharges from an electrode at the tip of a flexible probe, which is placed next to the stone. It is considered a highly effective means of bladder stone shattering and has become an accepted practice for this use. Since the shock waves generated during electro-hydraulic lithotripsy treatment are of sufficient force the probe must not be used 5 mm or closer to soft tissues otherwise severe damage will result. Since the discharge takes place within liquid phase the concretion is destroyed by virtue of combination of energy of the shock wave, caused by the discharge, hydraulic pressure of the surrounding liquid and collision of fragments in the liquid flow.

It can be easily appreciated that in lithotripsy the energy is transferred indirectly to the concretion via a liquid medium. Therefore the amount of energy required for fragmentation must be sufficient to overcome the strength of the concretion, to cause its fragmentation, after the energy has been delivered through the working liquid. For a concretion encased in a polymer matrix, even more additional energy will be needed. Unfortunately, release of such high levels of energy by producing shock waves might be harmful to the adjacent tissues and therefore potentially dangerous for the patient.

Another problem of almost all lithotriptors that are intended for destroying concretions by bringing mechanical energy of impact or shock wave is the fact that the stone is usually “displaced” with each pulse of energy, leaving the previous place and being “thrown” to another one. This displacement renders the operation complicated and may cause mechanical damage to the surrounding tissue. The instant invention addresses both of these problems.

### Selected Polymers and Methods of the Invention

The present invention improves significantly the success rate of lithotripsy and reduces the risk of tissue damage by forming a polymer plug behind a concretion (e.g., intracorporeal lithotripsy) prior to the fragmentation of the concretion. Importantly, the present invention prevents retropulsion fragments during lithotripsy.

The polymer plugs of the invention can be formed from viscous polymer compositions. In certain embodiments the viscous polymer composition is generated in situ, by one or more physical phenomena such as pH changes and/or ionic interactions. In other embodiments, the viscous polymer composition is generated ex vivo and then injected into the lumen of the mammal. In certain embodiments, the polymer plugs generated are non-tissue adhesive.

In certain embodiments, the polymer compositions of the invention comprise proteins selected from, for example, the group consisting of collagen, gelatin, elastin, albumin, protamine, fibrin, fibrinogen, keratin, reelin, caseine, or a mixture thereof. Other analogous proteins which can be used are well known to those of skill in the art.

In certain embodiment, the polymer compositions of the invention comprise hyaluronic acid or chitosan, or a mixture thereof.

In certain embodiments, the polymer compositions of the invention comprise synthetic materials selected from, for example, alginate, pectin, methylcellulose, carboxymethylcellulose, or a mixture thereof.

In certain embodiments, the polymers used in a methods of the invention are crosslinkable polymers. In one embodiment two solutions, a polymer solution and a crosslinker solution, are injected separately (e.g., through a dual lumen catheter) into a biological lumen wherein they gel, forming a polymer plug. Said polymer solution may comprise an anionic polymer, a cationic polymer or a non-ionically crosslinkable polymer. Such polymers may comprise one or more of the following: alginic acid, sodium alginate, potassium alginate, sodium gellan, potassium gellan, carboxymethylcellulose, hyaluronic acid, and polyvinyl alcohol. The cross-linking of the polymer to form a polymer plug may be achieved with anionic crosslinking ions, cationic crosslinking ions, or non-ionic crosslinking agents. Crosslinking agents include, but are not limited to, one or more of the following: phosphate, citrate, borate, succinate, maleate, adipate, oxalate, calcium, magnesium, barium and strontium. Exemplary pairings of polymers and crosslinkers include anionic polymer monomers with cations, such as, for example, alginates with calcium, barium or magnesium; gellans with calcium, magnesium or barium; or hyaluronic acid with calcium. An example of an exemplary pairing of a non-ionic polymer with a chemical crosslinking agent is a polyvinyl alcohol with borate (at a slightly alkaline pH).

One aspect of the present invention relates to a method of lithotripsy comprising the steps of:

injecting a first composition into a lumen of a mammal distal to a concretion, and optionally injecting a second composition into said lumen of a mammal distal to said concretion, wherein said second composition contacts said first composition, thereby forming a polymer plug; and

directing energy to said concretion causing the fragmentation of said concretion into a plurality of fragments.

In certain embodiments, the present invention relates to the aforementioned method, wherein said second composition is injected.

In certain embodiments, the present invention relates to the aforementioned method, wherein the distance from said concretion to said plug is between about 1 cm and about 5 cm.

In certain embodiments, the present invention relates to the aforementioned method, wherein the distance from said concretion to said plug is between about 2 cm and about 4 cm.

In certain embodiments, the present invention relates to the aforementioned method, wherein the distance from said concretion to said plug is about 3 cm.

In certain embodiments, the present invention relates to the aforementioned method, wherein said first composition is injected into said lumen through a percutaneous access device.

In certain embodiments, the present invention relates to the aforementioned method, wherein said first composition is injected into said lumen through a catheter or a syringe.

In certain embodiments, the present invention relates to the aforementioned method, wherein said second composition is injected into said lumen through a percutaneous access device.

In certain embodiments, the present invention relates to the aforementioned method, wherein said second composition is injected into said lumen through a catheter or a syringe.

In certain embodiments, the present invention relates to the aforementioned method, wherein the catheter is a dual lumen catheter or a triple lumen catheter.

In certain embodiments, the present invention relates to the aforementioned method, wherein the catheter is 1-10 French in size

In certain embodiments, the present invention relates to the aforementioned method, wherein the catheter is 1.5-3 French in size.

In certain embodiments, the present invention relates to the aforementioned method, wherein the catheter can be used to dispense one or more fluids other than, or in addition to, the polymer solution.

In certain embodiments, the present invention relates to the aforementioned method, wherein the syringe is a 1-100 cc syringe.

In certain embodiments, the present invention relates to the aforementioned method, wherein the syringe is a 1-50 cc syringe.

In certain embodiments, the present invention relates to the aforementioned method, wherein the syringe is a 1-5 cc syringe.

In certain embodiments, the present invention relates to the aforementioned method, wherein said injection of a first composition is done by hand or by an automated syringe pusher.

In certain embodiments, the present invention relates to the aforementioned method, wherein said injection of a second composition is done by hand or by an automated syringe pusher.

In certain embodiments, the present invention relates to the aforementioned method, wherein said energy is an acoustic shock wave, a pneumatic pulsation, an electrical hydraulic shock wave, or a laser beam.

In certain embodiments, the present invention relates to the aforementioned method, wherein said lumen is or is part of a kidney, a gall bladder, a ureter, a urinary bladder, a pancreas, a salivary gland, a small intestine or a large intestine.

In certain embodiments, the present invention relates to the aforementioned method, wherein said lumen is or is part of the ureter or kidney.

In certain embodiments, the present invention relates to the aforementioned method, wherein said concretion is a kidney stone, pancreatic stone, salivary stone, or biliary stone.

In certain embodiments, the present invention relates to the aforementioned method, wherein said concretion is a kidney stone.

In certain embodiments, the present invention relates to the aforementioned method, wherein said mammal is a human.

In certain embodiments, the present invention relates to the aforementioned method, wherein said first composition further comprises a contrast-enhancing agent.

In certain embodiments, the present invention relates to the aforementioned method, wherein said second composition further comprises a contrast-enhancing agent.

In certain embodiments, the present invention relates to the aforementioned method, wherein said contrast-enhancing agent is selected from the group consisting of radiopaque materials, paramagnetic materials, heavy atoms, transition metals, lanthanides, actinides, dyes, and radionuclide-containing materials.

In certain embodiments, the present invention relates to the aforementioned method, wherein said first composition comprises an anionic, cationic, or non-ionically crosslinkable polymer.

In certain embodiments, the present invention relates to the aforementioned method, wherein said first composition comprises collagen, gelatin, elastin, albumin, protamine, fibrin, fibrinogen, keratin, reelin, caseine, or a mixture thereof.

In certain embodiments, the present invention relates to the aforementioned method, wherein said first composition comprises hyaluronic acid or chitosan, or a mixture thereof.

In certain embodiments, the present invention relates to the aforementioned method, wherein said first composition comprises alginate, pectin, methylcellulose, carboxymethylcellulose, or a mixture thereof.

In certain embodiments, the present invention relates to the aforementioned method, wherein said first composition comprises alginic acid, sodium alginate, potassium alginate, sodium gellan, potassium gellan, carboxymethylcellulose, hyaluronic acid, polyvinyl alcohol, or a mixture thereof.

In certain embodiments, the present invention relates to the aforementioned method, wherein said second composition comprises a crosslinker selected from the group consisting of phosphate, citrate, borate, succinate, maleate, adipate, oxalate, calcium, magnesium, barium, strontium, or a combination thereof.

In certain embodiments, the present invention relates to the aforementioned method, wherein the concentration (w/w) of said crosslinker in said polymer plug in about 1% to about 0.005%.

In certain embodiments, the present invention relates to the aforementioned method, wherein the concentration (w/w) of said crosslinker in said polymer plug in about 0.5% to about 0.005%.

In certain embodiments, the present invention relates to the aforementioned method, wherein the concentration (w/w) of said crosslinker in said polymer plug in about 0.1% to about 0.005%.

In certain embodiments, the present invention relates to the aforementioned method, wherein said first composition comprises alginic acid, sodium alginate, potassium alginate, sodium gellan or potassium gellan; and said second composition comprises calcium, magnesium or barium.

In certain embodiments, the present invention relates to the aforementioned method, wherein said first composition comprises alginic acid, sodium alginate or potassium alginate; and said second composition comprises calcium.

In certain embodiments, the present invention relates to the aforementioned method, wherein said first composition comprises sodium gellan or potassium gellan; and said second composition comprises magnesium.

In certain embodiments, the present invention relates to the aforementioned method, wherein said first composition comprises hyaluronic acid; and said second composition comprises calcium.

In certain embodiments, the present invention relates to the aforementioned method, wherein said first composition comprises polyvinyl alcohol; and said second composition comprises borate.

### Kits of the Invention

This invention also provides kits for conveniently and effectively implementing the methods of this invention. Such kits comprise any of the compositions of the invention and a means for facilitating their use consistent with methods of this invention. Such kits provide a convenient and effective means for assuring that the methods are practiced in an effective manner. The compliance means of such kits includes any means which facilitates practicing a method of this invention. Such compliance means include instructions, packaging, and dispensing means, and combinations thereof. Kit components may be packaged for either manual or partially or wholly automated practice of the foregoing methods. In certain embodiments, the compositions of such a kit of the present invention are contained in one or more syringes, a compressible plastic or metal tube (for example, akin to a conventional toothpaste tube), or a packet that may be torn open.

### EXEMPLIFICATION

The invention now being generally described, it will be more readily understood by reference to the following prophetic examples, which are included merely for purposes of illustration of certain aspects and embodiments of the present invention, and are not intended to limit the invention.

### Example 1

The following experiment may be done to confirm the polymer plugs of the invention are effective in preventing stone migration during lithotripsy in an in vitro model

A plastic tube with an inner diameter of 0.9 cm can be selected to simulate the ureter. The tube can be partially filled with saline, and a human kidney stone (calcium oxalate) can be placed into the middle of the tube. A ureteroscope can be placed inside the tube close to the stone for visualization and the compositions or compositions of the invention can be injected into the tube through a standard single-lumen ureteral catheter placed through the working channel of the scope. The stone can be fragmented using either electro-hydraulic lithotripsy or laser lithotripsy.

### Example 2

The following experiment can be done to evaluate the time required to dissolve the polymer plugs of the invention using saline under static (worst-case) conditions in an in vitro model.

Prior to injection a composition of the invention may be made visible by addition of a small amount of Methylene Blue. After injection of the inventive composition into a Petri dish covered in saline at 37° C., the dissolution of the plug can be followed visually. Two different shapes of the plug can be used for the dissolution tests: a sphere, which has the least amount of surface area; and a string, which has the highest surface area and more precisely represents the shape of the polymer plug in the ureter. A 20 gauge syringe can be used to extrude the string of polymer onto the bottom of the Petri dish.

The Petri dish would not disturbed and every minute the Petri dish would be observed visually. Complete dissolution can be confirmed by swirling the Petri dish. The total time required for complete dissolution can be recorded.

### Example 3

The following experiment can be done in order to evaluate the time required to dissolve the polymer plugs of the invention in urine under static (worst-case) conditions in an in vitro model

Fresh urine samples could be obtained from a random sample of patients attending a urology clinic and the dissolution of polymer plugs of the invention, visualized by the addition of methylene blue, can be tested by injection the polymers plugs into a urine sample at 37° C. The time to dissolution can be recorded.

### Example 4

The following experiment can be done to confirm that the polymer plugs of the invention can be effectively dissolved and removed from the ureter (using saline irrigation) in an ex-vivo ureteral model.

Excised pig ureters (approx. 25 cm in length) can be fixed to a tray and the tray cab be submerged in a water bath heated to 37° C. A sheath can be inserted into the ureter, and a small (approximately 5 mm) simulated Plaster of Paris kidney stone can be placed in each ureter using a stone basket to advance the stone. A ureteroscope could then be placed in the ureter. A 3 F catheter can be advanced through the working channel of the scope approximately 3 cm beyond the stone. The compositions of the invention could be injected into the ureter through the catheter. For this experiment, methylene blue can be used to enhance visualization. A cystoscope can be used to visualize the catheter and the plug, allowing the tip of a catheter to be advanced into the plug. The site can be irrigated with either room temperature saline or cold water to dissolve and flush away polymer plug.

### Example 5

The following experiment can be done to confirm that the polymer plugs of the invention can be effectively dissolved and removed from the ureter (using saline irrigation) in vivo.

Adult female Yorkshire pigs could be anesthetized. In each animal, a supra-pubic incision could be made, the right ureter could be isolated, and a distal ureterotomy could be performed. A simulated Plaster of Paris kidney stone could be placed in the ureter about 2 to 3 cm above the ureterotomy. The size of the stone would be selected to be smaller than the ureter, placing it at risk for retropulsion. A semi-rigid ureteroscope could be passed through the ureter, the stone could be visualized, and a 3 F catheter could be passed through the working channel of the scope with the distal opening of the catheter beyond the stone. The compositions of the invention could be injected through the catheter to form a ureteral plug, then the catheter would then be removed. The stone could subsequently fragmented using an electro-hydraulic lithotripter. Cold saline can be used to dissolve the polymer plug and remove the stone fragments. Following lithotripsy and plug removal, the animals would be euthanized and the ureters could be surgically removed.

Pathological examination of the excised ureters would be performed by fixing the ureter in formalin. The tissue could be embedded in paraffin, sectioned transversely and stained with H&E. The tissue could then examined by a qualified pathologist.

### Example 6

The following experiment can be done to confirm that the polymer plug of the invention is effective in preventing stone migration following lithotripsy; to confirm that the material can be effectively removed; and to provide histological evaluation of the ureteral mucosa in a sub-chronic in vivo model.

Adult female Yorkshire pigs could be anesthetized. In each animal, a supra-pubic incision can be made, the right ureter can be isolated, and a distal ureterotomy can be performed. A simulated Plaster of Paris kidney stone measuring 3mm in diameter can be placed in the ureter about 2 to about 3 cm above the ureterotomy. The size of the stone would be selected to be smaller than the ureter, placing it at risk for retropulsion. A semi-rigid ureteroscope could be passed through the ureter, the stone could be visualized, and a 3F catheter could be passed through the working channel of the scope with the distal opening of the catheter approximately 2 cm beyond the stone. The compositions of the invention could be injected through the catheter to form a ureteral plug and the catheter would be removed. The stone can be subsequently fragmented using an electro-hydraulic lithotripter. As an alternative to flushing with cold saline, waiting for the polymer plug to start dissolving naturally could be tried.

Following lithotripsy and plug removal, the ureterotomies would be closed with fine absorbable sutures and the animals would be allowed to recover. After 1 week they can be anesthetized and through the same midline incision, the left ureter (control) and right ureter (experimental) could be transected and cannulated. Urine samples can be collected from each ureter. Urine/Plasma (UP) Creatinine, UP urea and fractional sodium excretions could be analyzed on timed urine collections and plasma could be analyzed using standard hospital laboratory methods. The values from the treated and control sides can be compared using an unpaired student's t-test.

Following collection of the urine and plasma samples, the kidneys and ureters would be harvested for pathologic examination and the animals would be euthanized. Pathological examination of the excised tissues could be performed by preserving the samples in formalin after which they would be embedded in paraffin, sectioned transversely, stained with H & E, and examined by a qualified pathologist.

### INCORPORATION BY REFERENCE

All of the U.S. patents and U.S patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of lithotripsy, comprising the steps of:
injecting a first composition into a lumen of a mammal distal to a concretion; thereby forming a polymer plug; and
directing energy to said concretion causing the fragmentation of said concretion into a plurality of fragments.

2. The method of claim 1, further comprising the step of:
injecting a second composition into said lumen of a mammal distal to said concretion, wherein said second composition contacts said first composition.

3. The method of claim 1, wherein the distance from said concretion to said plug is between about 1 cm and about 5 cm.

4. (canceled)

5. (canceled)

6. The method of claim 1, wherein said energy is an acoustic shock wave, a pneumatic pulsation, an electrical hydraulic shock wave, or a laser beam.

7. The method of claim 1, wherein said lumen is or is part of a kidney, a gall bladder, a ureter, a urinary bladder, a pancreas, a salivary gland, a small intestine or a large intestine.

8. (canceled)

9. The method of claim 1, wherein said concretion is a kidney stone, pancreatic stone, salivary stone, or biliary stone.

10. The method of claim 1, wherein said concretion is a kidney stone.

11. The method of claim 1, wherein said mammal is a human.

12. The method of claim 1, wherein said first composition further comprises a contrast-enhancing agent selected from the group consisting of radiopaque materials, paramagnetic materials, heavy atoms, transition metals, lanthanides, actinides, dyes, and radionuclide-containing materials.

13. The method of claim 2, wherein said second composition further comprises a contrast-enhancing agent selected from the group consisting of radiopaque materials, paramagnetic materials, heavy atoms, transition metals, lanthanides, actinides, dyes, and radionuclide-containing materials.

14. The method of claim 1, wherein said first composition comprises an anionic, cationic, or non-ionically crosslinkable polymer.

15. The method of claim 1, wherein said first composition comprises collagen, gelatin, elastin, albumin, protamine, fibrin, fibrinogen, keratin, reelin, caseine, or a mixture thereof.

16. The method of claim 1, wherein said first composition comprises hyaluronic acid or chitosan, or a mixture thereof.

17. The method of claim 1, wherein said first composition comprises alginate, pectin, methylcellulose, carboxymethylcellulose, or a mixture thereof.

18. The method of claim 1, wherein said first composition comprises alginic acid, sodium alginate, potassium alginate, sodium gellan, potassium gellan, carboxymethylcellulose, hyaluronic acid, polyvinyl alcohol, or a mixture thereof.

19. The method of claim 2, wherein said second composition comprises a crosslinker selected from the group consisting of phosphate, citrate, borate, succinate, maleate, adipate, oxalate, calcium, magnesium, barium, strontium, or a combination thereof.

20. The method of claim 2, wherein said first composition comprises alginic acid, sodium alginate, potassium alginate, sodium gellan or potassium gellan; and said second composition comprises calcium, magnesium or barium.

21. (canceled)

22. The method of claim 2, wherein said first composition comprises sodium gellan or potassium gellan; and said second composition comprises magnesium.

23. The method of claim 2, wherein said first composition comprises hyaluronic acid; and said second composition comprises calcium.

24. The method of claim 2, wherein said first composition comprises polyvinyl alcohol; and said second composition comprises borate.
